(19) United States
(12) Patent Application Publication
(10) Pub. No.: US 2020/0086003 A1
(43) Pub. Date: Mar. 19, 2020

(54) METHOD FOR DECELLULARIZATION OF SKIN TISSUE, METHOD FOR CONSTRUCTION OF ARTIFICIAL SKIN, METHOD FOR PREPARATION OF HYDROGEL OF DECELLULARIZED SKIN TISSUE, LYOPHILIZED, DECELLULARIZED SKIN TISSUE, AND BIOINK

(71) Applicant: POSTECH ACADEMY-INDUSTRY FOUNDATION, Pohang-si (KR)

(72) Inventors: Dong Woo CHO, Seoul (KR); Jeong Sik KONG, Bucheon-si (KR); Byoung Soo KIM, Gimcheon-si (KR)

(21) Appl. No.: 16/610,593

(22) PCT Filed: May 25, 2018
(86) PCT No.: PCT/KR2018/005948

§ 371 Date: Nov. 4, 2019

(30) Foreign Application Priority Data
May 31, 2017 (KR) 10-2017-0067934

(51) Int. Cl. A61L 27/36; C12N 5/071; A61L 27/38; A61L 27/52; A61L 27/60

(52) U.S. Cl. CPC A61L 27/3687; C12N 5/0698; C12N 5/0629; A61L 27/3691; A61L 2430/34; A61L 27/52; A61L 27/60; C12N 2509/00; A61L 2430/40; A61L 27/3886

(57) ABSTRACT

A method for decellularization of a skin tissue according to an embodiment of the present invention comprises: a step of preparing a skin tissue to be decellularized; a peeling preparation step of treating the skin tissue with a first solution containing trypsin; and a peeling step of removing subcutaneous fat from the skin tissue after the peeling preparation step.

### TECHNICAL FIELD

The research related to the present invention was carried out by the support of the ICT Convergence Original Technology Development Project (Project Title: Development and Commercialization of Artificial Skin Model Using 3D Bioprinting for Substitution of Animal Experiment, Project No.: 1711061192) under the supervision of the Ministry of Science and ICT.

The present invention relates to a method for decellularization of a skin tissue, a method for preparing artificial skin, a method for preparing a hydrogel of a decellularized skin tissue, a lyophilized decellularized skin tissue and bioink, and more specifically, a method for decellularization of a skin tissue for preparing artificial skin which is more similar to real skin, a method for preparing artificial skin, a method for preparing a hydrogel of a decellularized skin tissue, a lyophilized decellularized skin tissue and bioink.

### BACKGROUND ART

Currently, artificial skin is mostly prepared by encapsulating cells into a collagen hydrogel.

However, it is difficult to handle artificial skin prepared from a collagen hydrogel because it is very fragile due to the weak physical property of artificial skin. In addition, when artificial skin is incubated for a long period of time, its volume will decrease significantly and at the same time the cells will die, and thus cannot be used after a certain period of time.

Furthermore, since the dermal layer of skin is not composed of collagen alone, artificial skin prepared using a collagen hydrogel is still limited in creating an environment similar to real skin.

### DISCLOSURE

### Technical Problem

The objects of the present invention to achieve are to provide a method for decellularization of a skin tissue for preparing artificial skin which is more similar to real skin, a method for preparing artificial skin, a method for preparing a hydrogel of a decellularized skin tissue, a lyophilized decellularized skin tissue and bioink.

The objects of the present invention are not limited to those described above, and other objects not described above will be clearly understood by those skilled in the art from the following description.

### Technical Solution

The method for decellularization of a skin tissue according to an embodiment of the present invention to solve the above objects includes a step of preparing a skin tissue to be decellularized, a peeling preparation step of treating the skin tissue with a first solution containing trypsin, and a peeling step of removing subcutaneous fat from the skin tissue after the peeling preparation step.

The first solution may further include ethylenediaminetetraacetic acid (EDTA).

The first solution may be one in which 1 mM or less of the ethylenediaminetetraacetic acid (EDTA) is dissolved and 0.25% or less of the trypsin is dissolved.

The epidermis of the skin tissue may be removed in at least one step of the peeling preparation step and the peeling step.

The method may further include a DNA treatment step in which the skin tissue, after undergoing the peeling step, is treated using a second solution that includes magnesium ions and DNase.

The second solution may further include, in a buffer solution, 10 mM or less of MgCl₂ and 30 U/mL or less of DNase.

The buffer solution may include phosphate buffered saline (PBS) and the DNA treatment step may be performed at a temperature between 30° C. or higher and 40° C. or lower for at least 20 hours.

The method may further include a cell removal step in which the skin tissue, after undergoing the peeling step, is treated using a buffer solution that includes ethylenediaminetetraacetic acid (EDTA) and a non-ionic surfactant.

The buffer solution may be phosphate buffered saline (PBS) in which 25 mM or less of the ethylenediaminetetraacetic acid (EDTA) and 1% or less of the non-ionic surfactant are dissolved.

The cell removal step may be performed at a temperature between 30° C. or higher and 40° C. or lower for at least 20 hours.

The decellularized skin tissue according to an embodiment of the present invention to achieve the above objects may be a decellularized skin tissue which is lyophilized for at least 40 hours.

The bioink according to an embodiment of the present invention to achieve the above objects may be one in which normal human dermal fibroblasts (NHDF) are mixed with a decellularized skin tissue.

The method for preparing artificial skin according to an embodiment of the present invention to achieve the above objects includes seeding normal human epithelial keratinocytes (NHEK) to a bioink, followed by culturing the same.

The method for preparing a hydrogel of a decellularized skin tissue according to an embodiment of the present invention to achieve the above objects includes dissolving a decellularized skin tissue along with pepsin in an acetic acid solution to prepare a pre-gel; and neutralizing the pre-gel.

Other specific details of the invention are included in the Detailed Description and Drawings.

### Advantageous Effects

According to the embodiments, the present invention has at least the following effects.

It is possible to prepare artificial skin that closely resembles a real skin.

The effects according to the present invention are not limited by the contents illustrated above and more various effects are included in the present specification.

### DESCRIPTION OF DRAWINGS

FIG. 1 shows a flow chart illustrating a method for decellularization of a skin tissue according to an embodiment of the present invention.

FIG. 2 shows a graph comparing the contents of collagen, glycosaminoglycans, elastin, hyaluronic acid, and DNA, between those contained in a decellularized skin tissue (D-dECM), which is prepared by a method for decellularization according to an embodiment of the present invention, and those originally contained in a skin tissue (native), using a biochemical assay.

FIG. 3 shows a flow chart illustrating a method for preparing a hydrogel of a decellularized skin tissue according to an embodiment of the present invention.

FIGS. 4 to 6 show graphs comparing physical properties between a hydrogel, which is prepared by a method for preparing a hydrogel of a decellularized skin tissue according to an embodiment of the present invention, and a collagen hydrogel.

FIG. 7 shows a scanning electron microscope (SEM) image of D-dECM hydrogel (D-dECM), which was prepared by a method for preparing a hydrogel of a decellularized skin tissue according to an embodiment of the present invention, and that of a collagen hydrogel (COL).

FIG. 8 shows images comparing the growth state of NHDF and NHEK, which were each cultured in a hydrogel, which is prepared by a method for preparing a hydrogel of a decellularized skin tissue according to an embodiment of the present invention, and in a collagen hydrogel.

FIG. 9 shows a graph illustrating qRT-PCR data of NHDF, which was cultured in a hydrogel, which is prepared by a method for preparing a hydrogel of a decellularized skin tissue according to an embodiment of the present invention, and in a collagen hydrogel, respectively.

FIG. 10 shows images comparing and illustrating the cross-sections of artificial skin made of a hydrogel, which is prepared by a method for preparing a hydrogel of a decellularized skin tissue according to an embodiment of the present invention, and artificial skin made of a collagen hydrogel.

FIG. 11 shows graphs comparing and illustrating the thickness and width of artificial skin made of a hydrogel, which is prepared by a method for preparing a hydrogel of a decellularized skin tissue according to an embodiment of the present invention, and artificial skin made of a collagen hydrogel.

FIG. 12 shows images comparing and illustrating the measurement results of skin contact angle of artificial skin prepared using collagen hydrogel alone (Only COL), artificial skin prepared based on a collagen hydrogel (Skin-C), artificial skin prepared based on a hydrogel of the decellularized skin tissue according to an embodiment of the present invention alone (Only D-dECM), and artificial skin prepared based on a hydrogel of the decellularized skin tissue according to an embodiment of the present invention (Skin-D).

FIG. 13 shows a graph comparing and illustrating the measurement results of the electrical resistance between skin epidermis of artificial skin prepared using collagen hydrogel alone (Only COL), artificial skin prepared based on a collagen hydrogel (Skin-C), artificial skin prepared based on a hydrogel of the decellularized skin tissue according to an embodiment of the present invention alone (Only D-dECM), and artificial skin prepared based on a hydrogel of the decellularized skin tissue according to an embodiment of the present invention (Skin-D).

FIG. 14 shows a graph comparing and illustrating the test results of skin barrier permeability of artificial skin prepared using collagen hydrogel alone (Only COL), artificial skin prepared based on a collagen hydrogel (Skin-C), artificial skin prepared based on a hydrogel of the decellularized skin tissue according to an embodiment of the present invention alone (Only D-dECM), and artificial skin prepared based on a hydrogel of the decellularized skin tissue according to an embodiment of the present invention (Skin-D).

### MODE FOR INVENTION

Advantages and features of the present invention, and methods for accomplishing the same will become apparent when referred to the embodiments described below in detail in conjunction with the accompanying drawings. However, the present invention is not limited to the embodiments disclosed below, but may be implemented in various different forms, and the embodiments are provided only to make the disclosure of the present invention complete, and to fully deliver the scope of the invention to those skilled in the art, and the invention is only defined by the scope of the claims. Like reference numerals refer to like elements throughout the specification.

In addition, the embodiments described herein will be described with reference to cross-sectional and/or schematic views, which are ideal illustrations of the invention. Accordingly, shapes of the exemplary views may be modified by manufacturing techniques and/or tolerances. In addition, each element in each drawing shown in the present invention may be shown to be somewhat enlarged or reduced in view of the convenience of description. Like reference numerals refer to like elements throughout the specification.

Hereinafter, the present invention will be described with reference to the drawings for explaining a method for decellularization of a skin tissue according to an embodiment of the present invention, a decellularized skin tissue using the same, a method for preparing a hydrogel of a decellularized skin tissue, a method for preparing artificial skin, etc.

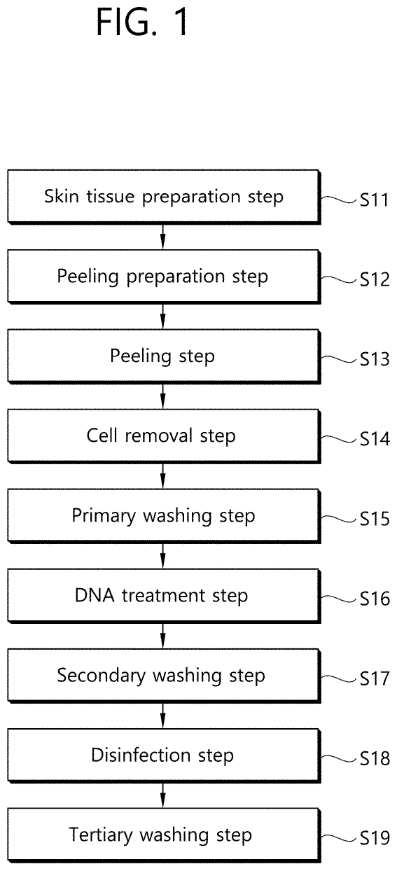

FIG. 1 shows a flow chart illustrating a method for decellularization of a skin tissue according to an embodiment of the present invention.

As illustrated in FIG. 1, the method for decellularization of a skin tissue according to an embodiment of the present invention includes a skin tissue preparation step (S11), a peeling preparation step (S12), a peeling step (S13), a cell removal step (S14), a primary washing step (S15), a DNA treatment step (S16), a secondary washing step (S17), a disinfection step (S18), and a tertiary washing step (S19).

In the skin tissue preparation step (S11), a skin tissue to be decellularized is prepared. In the method for decellularization according to an embodiment of the present invention, a pig skin tissue is used as the skin tissue to be decellularized. It is desirable that the pig skin tissue be prepared to include a dermal layer.

In the peeling preparation step (S12), a solution (a first solution), in which trypsin at a concentration of about 0.25% is added to an ethylenediaminetetraacetic acid (EDTA) solution at a concentration of about 1 mM, may be used. According to embodiments, various concentrations of ethylenediaminetetraacetic acid and trypsin may be selected.

In the peeling preparation step (S12), the pig skin tissue prepared in Step S11 may be treated with the first solution for about 6 hours. The pig skin tissue may be immersed in the first solution for 6 hours or stirred along with the first solution. During the treatment of the pig skin tissue, at least part of the epidermal layer of the skin tissue may be degraded by the first solution.

In the peeling step (S13), subcutaneous fat is removed from the pig skin tissue that underwent Step S12. In addition, the epidermal layer remaining without being removed from Step S12 is also removed.

During the progress of Step S12, the subcutaneous fat and epidermis of the skin tissue become flabby by the first solution. In the peeling step (S13), subcutaneous fat and epidermis may be removed by physical methods, such as cutting or scraping with a tool (e.g., knives). In the peeling step (S13), all except the dermal layer of the skin tissue can be removed.

In the cell removal step (S14), the pig skin tissue which underwent Step S13 with a solution where ethylenediaminetetraacetic acid (EDTA) and a surfactant are dissolved (a cell removal solution) in a buffer solution, can be treated.

In this embodiment, phosphate buffered saline (PBS) was used as a buffer solution, and a non-ionic surfactant (Triton X-100) was used as a surfactant. The phosphate buffered saline (PBS) was contained at a concentration of about 25 mM and Triton X-100 was contained at a concentration of about 1%, and the treatment was performed at a temperature between 30° C. or higher and 40° C. or lower for at least 20 to 30 hours. Preferably, the treatment can be performed under the atmosphere at about 37° C. for about 24 hours.

In the cell removal step (S14), the skin tissue may be treated in an immersed state in the cell removing solution or may be treated by stirring along with the cell removal solution. During the progress of the cell removal step (S14), the cellular materials in the skin tissue are removed or degraded due to the activity of surfactants, etc.

In the primary washing step (S15), the skin tissue which underwent Step S14 is washed with a buffer solution. In this embodiment, the skin tissue was washed by stirring along with phosphate buffered saline (PBS) for about 14 hours. Through the primary washing step (S15), most of the cellular materials which are degraded in Step S14 and remain in the tissue are washed and removed from the tissue.

In the DNA treatment step (S16), DNA is digested in a skin tissue which underwent Step S15. In this embodiment, the DNA in the skin tissue was digested using a solution (a second solution) where about 10 mM MgCl₂ and 30 U/mL of DNase were included in phosphate buffered saline (PBS).

The DNA treatment step (S16) was performed at a temperature between 30° C. or higher and 40° C. or lower for at least 20 to 30 hours. Preferably, the treatment can be performed under the atmosphere at about 37° C. for about 24 hours. In the DNA treatment step (S16), the skin tissue may be treated in an immersed state in the cell removing solution or may be treated by stirring along with the second solution.

During the progress of DNA treatment step (S16), the DNA in the skin tissue is digested by DNase. In addition, since the second solution according to an embodiment of the present invention contains about 10 mM MgCl₂, the digestion of DNA is proceeded more effectively. This is because magnesium ions contained in the second solution cut the DNA into small pieces and provide an environment where DNase can easily digest the DNA.

In the secondary washing step (S17), the skin tissue which underwent Step S16 is washed with a buffer solution. In this embodiment, the skin tissue was washed by stirring along with phosphate buffered saline (PBS) for about 48 hours. Through the secondary washing step (S17), most of the materials which are degraded in Step S16 and remain in the tissue are washed and removed from the tissue.

In addition, in the secondary washing step (S17), the skin tissue which was washed with phosphate buffered saline (PBS) for about 48 hours can be washed again with distilled water. In this embodiment, the skin tissue which was washed with phosphate buffered saline (PBS) for about 48 hours was washed again with distilled water 3 times for 15 minutes in each wash. The washing time and number of washing with distilled water may vary depending on the embodiment.

In the disinfection step (S18), the skin tissue which underwent Step S17 with a disinfection solution is sterilized. As the disinfection solution, any solution containing peracetic acid and ethanol that can destroy the cell wall of microorganisms may be used. In this embodiment, a solution in which about 4% ethanol and about 0.1% peracetic acid are dissolved in distilled water was used as the disinfection solution. The disinfection step (S18) can be performed for about at least 2 hours.

In the tertiary washing step (S19), the skin tissue which underwent Step S18 with a buffer solution is washed. In this embodiment, after washing twice the skin tissue by stirring along with phosphate buffered saline (PBS) in the tertiary washing step (S19) for about 15 minutes in each wash, the skin tissue was washed twice by stirring along with deionized water for about 15 minutes in each wash.

Through the above-described steps (S11-S19), a decellularized skin tissue, and more specifically dermis decellularized extracellular matrix (D-dECM), can be obtained.

The decellularized skin tissue (D-dECM) may be stored/used after lyophilization for over 40 hours.

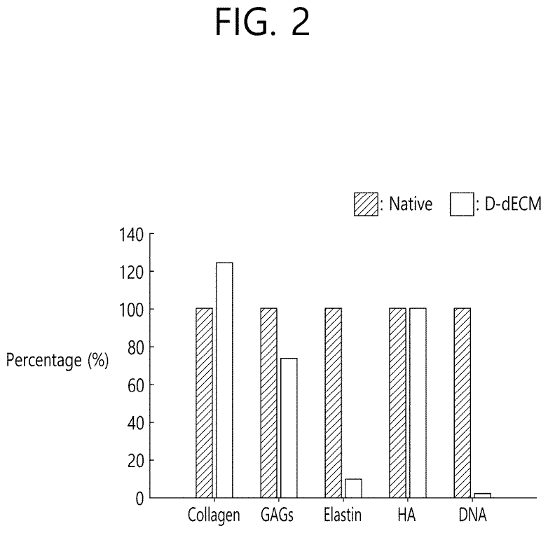

FIG. 2 shows a graph comparing the contents of collagen, glycosaminoglycans, elastin, hyaluronic acid, and DNA, between those contained in a decellularized skin tissue (D-dECM), which is prepared by a method for decellularization according to an embodiment of the present invention, and those originally contained in a skin tissue (native), using a biochemical assay.

As illustrated in FIG. 2, the decellularized skin tissue (D-dECM), prepared by the method for decellularization according to an embodiment of the present invention, shows that about 125% of collagen (Collagen), about 74% of glycosaminoglycans (GAGs), about 9.8% of elastin, about 2.3% of DNA, and about 100% of hyaluronic acid (HA) are contained, compared to the original skin tissue (native)—a pig skin tissue.

Accordingly, it can be confirmed that the method for decellularization according to an embodiment of the present invention can preserve glycosaminoglycans (GAGs), collagen (Collagen), and hyaluronic acid (HA) while effectively removing cells.

Meanwhile, since hyaluronic acid can attach to various extracellular matrices to stabilize the extracellular matrices and promote cell migration during wound healing; and low molecular weight hyaluronic acid can cause blood vessel formation(angiogenesis), the decellularized skin tissue (D-dECM) prepared by the method for decellularization according to an embodiment of the present invention can be effectively used in preparing a therapeutic agent for wound healing.

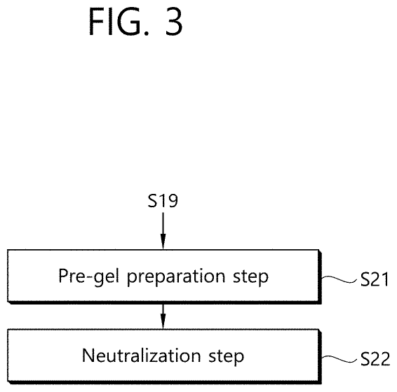

FIG. 3 shows a flow chart illustrating a method for preparing a hydrogel of a decellularized skin tissue according to an embodiment of the present invention.

As illustrated in FIG. 3, the method for preparing a hydrogel of a decellularized skin tissue according to an embodiment of the present invention further includes a pre-gel preparation step (S21) and a neutralization step (S22), in addition to the above-described decellularization method.

In the pre-gel preparation step (S21), a decellularized skin tissue (D-dECM) is dissolved in an acetic acid solution along with pepsin to prepare a D-dECM pre-gel.

In the pre-gel preparation step (S21), the decellularized skin tissue (D-dECM) can be lyophilized for at least 40 hours. In this embodiment, the D-dECM pre-gel was prepared by dissolving the lyophilized D-dECM in a solution, in which pepsin with about 10% weight of that of the lyophilized D-dECM was dissolved in an about 0.5 M acetic acid solution.

In the neutralization step (S22), the acidic D-dECM pre-gel is neutralized. In the neutralization step (S22), the pH of the D-dECM pre-gel can be adjusted to a neutral pH using a basic solution.

In this embodiment, a typical basic solution, NaOH, was used, and more specifically, the pH of the acidic D-dECM pre-gel was adjusted to a neutral pH with a 10 M NaOH solution.

The neutralization step (S22) may be also performed using a different basic solution depending on the embodiment.

The D-dECM, when the method is proceeded to Step S22, will become in a state of hydrogel.

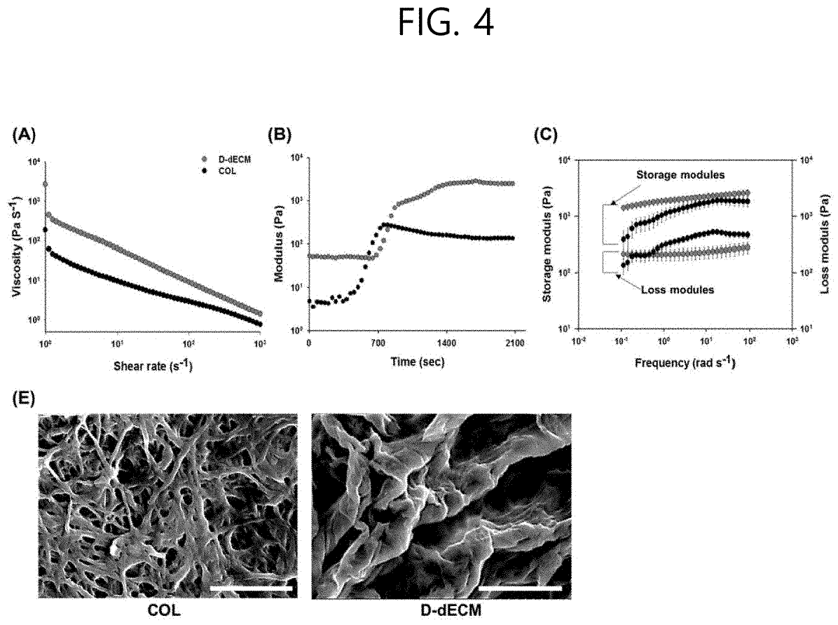

FIGS. 4 to 6 show graphs comparing physical properties between a hydrogel, which is prepared by a method for preparing a hydrogel of a decellularized skin tissue according to an embodiment of the present invention, and a collagen hydrogel.

The graph illustrated in FIG. 4 compares and illustrates the correlation of shear rate and viscosity between the D-dECM hydrogel (D-dECM) and the collagen hydrogel (COL). As illustrated in FIG. 4, the D-dECM hydrogel (D-dECM) shows a shear thinning behavior and thus crosslinking occurs by heat. Therefore, injection by pressure is possible thus enabling printing via 3D printing.

In addition, the D-dECM hydrogel may be mixed with cells, and due to the neutral pH of the D-dECM hydrogel, the cells will not be destroyed even when the cells are mixed with the D-dECM hydrogel. The D-dECM hydrogel can be crosslinked by heat alone without any other chemical treatment, it is possible to prevent adverse effects on cell activity by chemicals during a hardening process.

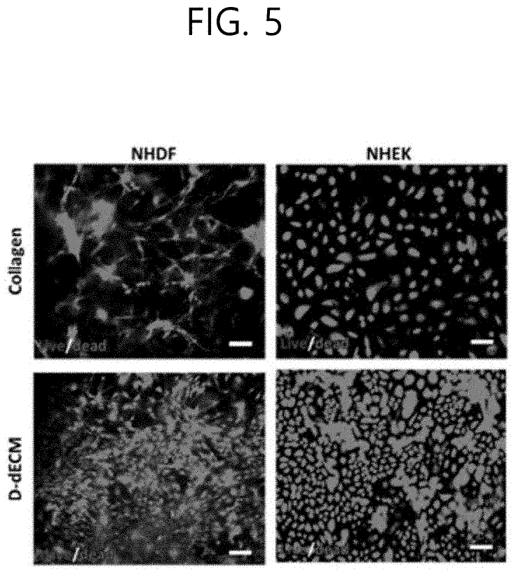

The graph illustrated in FIG. 5 compares and illustrates the complex modulus with time between the D-dECM hydrogel (D-dECM) and the collagen hydrogel (COL). As illustrated in FIG. 5, it can be confirmed that the complex modulus of the D-dECM hydrogel (D-dECM) is about 10 times that of the collagen hydrogel (COL), and this means that the strength of the D-dECM hydrogel (D-dECM) is about 10 times greater compared to that of the collagen hydrogel (COL).

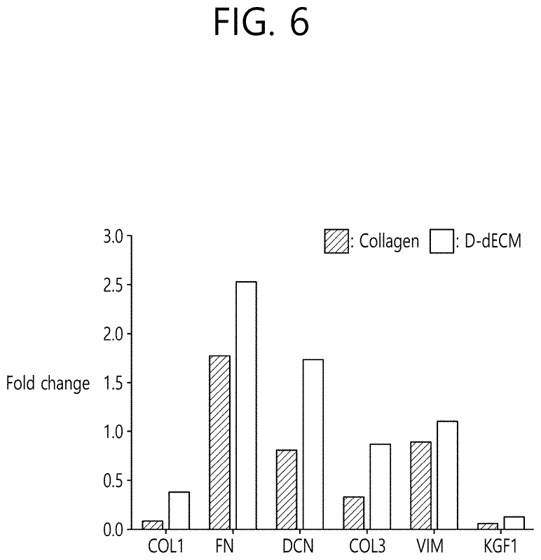

The graph illustrated in FIG. 6 compares and illustrates the correlation of storage modulus and loss modulus between the D-dECM hydrogel (D-dECM) and the collagen hydrogel (COL). As illustrated in FIG. 6, the D-dECM hydrogel (D-dECM) has a greater storage modulus than that of the collagen hydrogel (COL), whereas the overall loss modulus of the D-dECM hydrogel (D-dECM) is lower than that of the collagen hydrogel (COL).

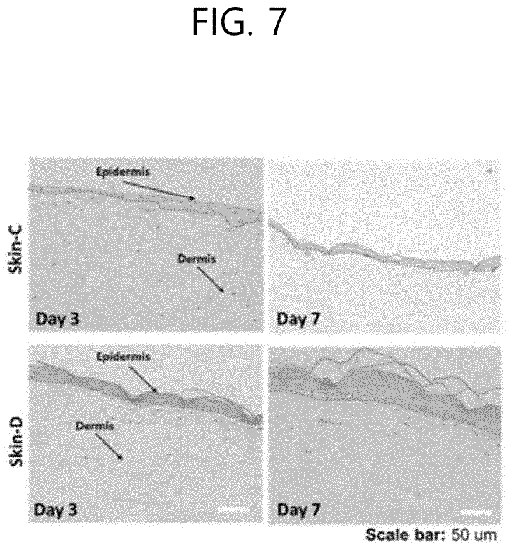

FIG. 7 shows a scanning electron microscope (SEM) image of D-dECM hydrogel (D-dECM), which was prepared by a method for preparing a hydrogel of a decellularized skin tissue according to an embodiment of the present invention, and that of a collagen hydrogel (COL).

The greater strength of the D-dECM hydrogel (D-dECM) compared to that of the collagen hydrogel (COL) is expected, as illustrated in FIG. 7, the collagen bundle of the D-dECM hydrogel (D-dECM) is thicker than that of the hydrogel (COL), and that hyaluronic acid or glycosaminoglycans (GAGs) have increased their ability to retain water, thereby increasing their ability to resist external pressure.

Meanwhile, [Table 1] is the analysis results of the contents of various growth factors contained in the D-dECM hydrogel and the collagen hydrogel performed via LC-MS/MS mass spectrometry.

TABLE 1

| (pg/mL) | D-dECM | COL |
|---|---|---|
| AR | 288.3 | 96.4 |
| BDNF | 30.6 | 28.8 |
| bFGF | 617.2 | 285.2 |
| BMP-4 | 1413.3 | 282.9 |
| BMP-5 | 3351.1 | 2174.2 |
| BMP-7 | 1431.7 | 1183.2 |
| b-NGF | 21.6 | 41.9 |
| EGF | 2.9 | 0.2 |
| EGF R | 209.3 | 67.6 |
| EG-VEGF | 52.1 | 38.6 |
| FGF-4 | 3863.8 | 1025.3 |
| FGF-7 | 233.2 | 77.0 |
| GDF-15 | 23.6 | 17.1 |
| GDNF | 102.2 | 24.7 |
| GH | 417.0 | 236.8 |
| HB-EGF | 68.1 | 53.2 |
| HGF | 126.6 | 57.5 |
| IGFBP-1 | 133.5 | 64.0 |
| IGFBP-2 | 1691.2 | 606.5 |
| IGFBP-3 | 5223.7 | 5552.3 |
| IGFBP-4 | 8114.3 | 2820.7 |
| IGFBP-6 | 2766.8 | 1129.3 |
| IGF-I | 1521.1 | 0.0 |
| Insulin | 379.7 | 945.8 |
| MCSF R | 885.0 | 408.3 |
| NGF R | 395.0 | 234.4 |
| NT-3 | 228.8 | 151.5 |
| NT-4 | 176.0 | 128.0 |
| OPG | 84.6 | 44.1 |
| PDGF-AA | 61.6 | 23.1 |
| PIGF | 91.3 | 83.7 |
| SCF | 134.7 | 74.3 |
| SCF R | 520.1 | 199.2 |
| TGFa | 158.2 | 44.7 |
| TGFb1 | 4584.6 | 6715.9 |
| TGFb3 | 257.6 | 171.5 |
| VEGF | 373.6 | 254.2 |
| VEGF R2 | 342.8 | 309.5 |
| VEGF R3 | 385.6 | 271.0 |
| VEGF-D | 282.1 | 299.9 |

As illustrated in Table 1, it can be confirmed that most of the growth factors that promote the growth differentiation ability of cells are present more in the D-dECM hydrogel than in the collagen hydrogel.

Meanwhile, the D-dECM hydrogel according to an embodiment of the present invention can be mixed with human cells and used as a bioink. A bioink can be used as a culture material for tissue/organ mimics or as an ink for 3D cell printing.

In particular, artificial skin may be prepared by culturing a bioink in which normal human dermal fibroblasts (NHDF) and/or normal human epithelial keratinocytes (NHEK) are mixed with the D-dECM hydrogel according to an embodiment of the present invention.

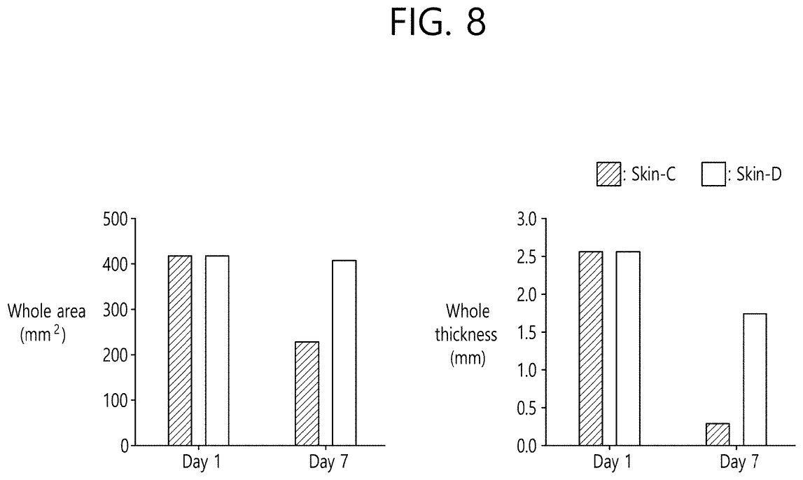

FIG. 8 shows images comparing the growth state of NHDF and NHEK, which were each cultured in a hydrogel, which is prepared by a method for preparing a hydrogel of a decellularized skin tissue according to an embodiment of the present invention, and in a collagen hydrogel.

The images illustrated in FIG. 8 are photographed images after encapsulating NHDF to a hydrogel (D-dECM) prepared by the method for preparing a hydrogel of a decellularized skin tissue according to an embodiment of the present invention and to the collagen hydrogel (Collagen) followed by culturing the same for two weeks, respectively; and photographed images after seeding NHEK to a hydrogel (D-dECM) prepared by the method for preparing a hydrogel of a decellularized skin tissue according to an embodiment of the present invention and to the collagen hydrogel (Collagen) followed by culturing the same for two weeks.

As illustrated in FIG. 8, it can be confirmed that the D-dECM, to which NHDF and NHEK were encapsulated or seeded, showed a higher growth rate and a higher rate of cells that are attached to one another, compared to the collagen hydrogel (Collagen), to which NHDF and NHEK were encapsulated or seeded.

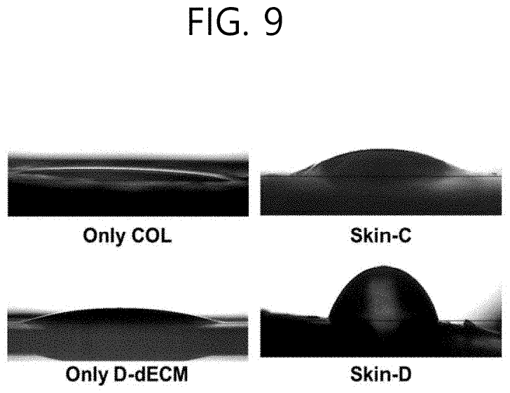

FIG. 9 shows a graph illustrating qRT-PCR data of NHDF, which was cultured in a hydrogel, which is prepared by a method for preparing a hydrogel of a decellularized skin tissue according to an embodiment of the present invention, and in a collagen hydrogel, respectively.

As illustrated in FIG. 9, it can be confirmed that the RNA expression levels of collagen type1 (COL1), fibronectin (FN), decorin (DCN), collagen type3 (COL3), vimentin (VIM), and KGF in the NHDF, which is encapsulated into the D-dECM, are higher. Accordingly, it can be confirmed that D-dECM hydrogel is more helpful for NHDF activity than the collagen hydrogel.

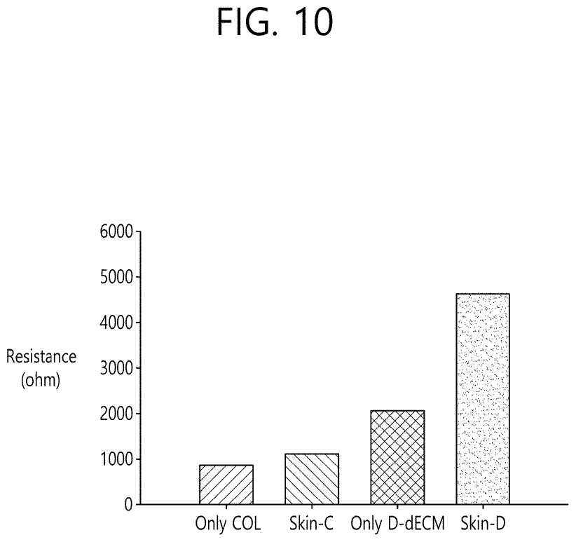

FIG. 10 shows images comparing and illustrating the cross-sections of artificial skin (Skin-D) made of a hydrogel, which is prepared by a method for preparing a hydrogel of a decellularized skin tissue according to an embodiment of the present invention, and artificial skin (Skin-C) made of a collagen hydrogel.

As illustrated in FIG. 10, it was confirmed that in the artificial skin (Skin-D) using the D-dECM hydrogel, the stratum corneum was formed within 3 days and the epidermis grew to 80 μm within a week and thus that the thickness (approximately 0.06 mm to 0.2 mm) was similar to that of the epidermis of the real skin. This means that the use of the D-dECM hydrogel can significantly shorten the time required to prepare histological artificial skin compared to use of the collagen hydrogel.

Further, comparing the dermis, it can be confirmed that the artificial skin (Skin-D) prepared using the D-dECM hydrogel shows that the amount of collagen type 1 self-produced by NHDF is greater. This means that the artificial skin (Skin-D) using the D-dECM hydrogel is similar to real human skin, compared to the artificial skin (Skin-C) prepared using the collagen hydrogel. Accordingly, the D-dECM hydrogel can be used as an agent for skin transplantation or wound treatment.

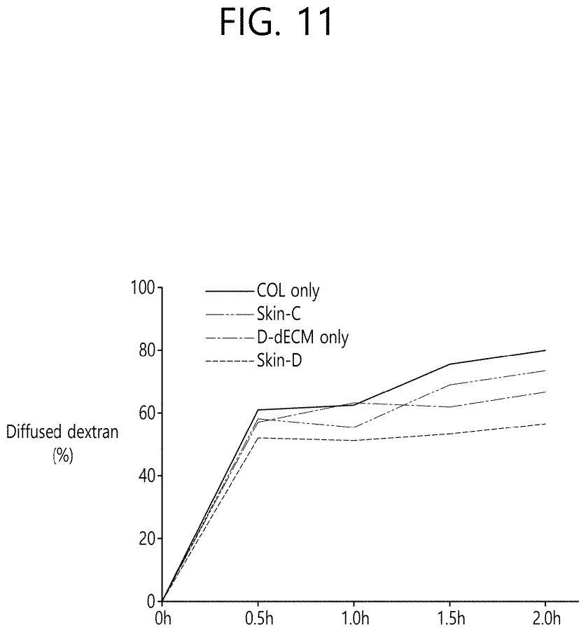

FIG. 11 shows graphs comparing and illustrating the thickness and width of artificial skin (Skin-D) made of a hydrogel, which is prepared by a method for preparing a hydrogel of a decellularized skin tissue according to an embodiment of the present invention, and artificial skin (Skin-C) made of a collagen hydrogel.

As illustrated in FIG. 11, it can be confirmed that the artificial skin (Skin-C) prepared using the collagen hydrogel showed a drastic decrease both in whole area and whole thickness on the 7^{th} day, whereas the artificial skin (Skin-D) prepared using the D-dECM hydrogel maintained whole area without a noticeable change and showed a slight decrease in whole thickness but the degree of decrease was significantly less compared to the artificial skin (Skin-C) prepared using the collagen hydrogel.

Accordingly, in the case of performing the experiment while culturing artificial skin for a long period of time, it is more advantageous to culture artificial skin using the D-dECM hydrogel than using the collagen hydrogel.

Meanwhile, in the case of preparing artificial skin with the collagen hydrogel, encapsulating NHDF into the collagen hydrogel and awaiting the collagen hydrogel to shrink, NHEK was seeded onto the surface of the stabilized collagen hydrogel.

However, in the case of the D-dECM hydrogel according to an embodiment of the present invention, it is possible to immediately seed NHEK without awaiting the shrinking of the D-dECM hydrogel after encapsulating NHDF into the D-dECM hydrogel.

In the case of preparing artificial skin using the D-dECM hydrogel according to an embodiment of the present invention, it is not required to await shrinking of the D-dECM hydrogel after encapsulating NHDF thereinto, the preparation time can be shortened compared to preparation of the artificial skin with the conventional collagen hydrogel.

Hereinafter, the measurement results of similarity between the artificial skin prepared using the D-dECM hydrogel according to an embodiment of the present invention and the real skin will be described.

FIG. 12 shows images comparing and illustrating the measurement results of skin contact angle of artificial skin prepared using collagen hydrogel alone (Only COL), artificial skin prepared based on a collagen hydrogel (Skin-C), artificial skin prepared based on a hydrogel of the decellularized skin tissue according to an embodiment of the present invention alone (Only D-dECM), and artificial skin prepared based on a hydrogel of the decellularized skin tissue according to an embodiment of the present invention (Skin-D).

The actual contact angle of the skin is known to be 70° to 80°.

While the contact angle of the artificial skin (Skin-C) prepared based on the collagen hydrogel was measured to be 35.6°, the contact angle of the artificial skin (Skin-D) prepared based on the D-dECM hydrogel was measured to be at 66.3°.

Accordingly, it is thought that the surface hydrophobicity of the artificial skin (Skin-D) prepared based on the D-dECM hydrogel is more similar to that of real skin, compared to the artificial skin (Skin-C) prepared based on the collagen hydrogel.

FIG. 13 shows a graph comparing and illustrating the measurement results of the electrical resistance between skin epidermis of artificial skin prepared using collagen hydrogel alone (Only COL), artificial skin prepared based on a collagen hydrogel (Skin-C), artificial skin prepared based on a hydrogel of the decellularized skin tissue according to an embodiment of the present invention alone (Only D-dECM), and artificial skin prepared based on a hydrogel of the decellularized skin tissue according to an embodiment of the present invention (Skin-D).

The electrical resistance between epidermis in the epithelium of real skin is known to be about 5,500Ω.

As illustrated in FIG. 13, while the electrical resistance between epidermis of the artificial skin (Skin-D) prepared based on the D-dECM hydrogel was measured to be 4,616.7Ω, the electrical resistance between epidermis of the artificial skin (Skin-C) prepared based on the collagen hydrogel was measured to be even lower than that of the artificial skin (Only D-dECM), which was prepared based on the D-dECM hydrogel alone.

Accordingly, even in the character with regard to electrical resistance between epidermis, the artificial skin (Skin-D) prepared based on the D-dECM hydrogel was more similar to the real skin than the artificial skin (Skin-C) prepared based on the collagen hydrogel.

FIG. 14 shows a graph comparing and illustrating the test results of skin barrier permeability of artificial skin prepared using collagen hydrogel alone (Only COL), artificial skin prepared based on a collagen hydrogel (Skin-C), artificial skin prepared based on a hydrogel of the decellularized skin tissue according to an embodiment of the present invention alone (Only D-dECM), and artificial skin prepared based on a hydrogel of the decellularized skin tissue according to an embodiment of the present invention (Skin-D).

As illustrated in FIG. 14, even in the dextran permeability test, it was confirmed that the artificial skin (Skin-D) prepared based on the D-dECM hydrogel showed about 1.5 fold greater prevention of dextran permeability than the artificial skin (Skin-C) prepared based on the collagen hydrogel, thus showing greater functional performance as a skin barrier.

Those skilled in the art will appreciate that the present invention can be embodied in other specific forms without altering the technical spirit or essential features of the present invention. Therefore, it should be understood that the embodiments described above are exemplary and not restrictive in all respects. The scope of the present invention is illustrated by the following claims rather than the detailed description, and all changes or modifications derived from the meaning and scope of the claims and their equivalents should be construed as being included in the scope of the present invention.

### MODE FOR CARRYING OUT THE INVENTION

The method for decellularization of a skin tissue according to an embodiment of the present invention includes a step of preparing a skin tissue to be decellularized; a peeling preparation step of treating the skin tissue with a first solution containing trypsin; and a peeling step of removing subcutaneous fat from the skin tissue after the peeling preparation step.

1. A bioink in which normal human dermal fibroblasts (NHDF) are mixed with a decellularized skin tissue by a method for decellularization of a skin tissue, which comprises the steps of:
a step of preparing a skin tissue to be decellularized;
a peeling preparation step of treating the skin tissue with a first solution containing trypsin; and
a peeling step of removing subcutaneous fat from the skin tissue after the peeling preparation step;
a cell removal step in which the skin tissue, after undergoing the peeling step, is treated using a buffer solution that comprises ethylenediaminetetraacetic acid (EDTA) and a non-ionic surfactant;
a primary washing step in which the skin tissue, which has undergone the cell removal step, is washed;
a DNA treatment step in which the skin tissue, which has undergone the primary washing step, is treated using a second solution that comprises magnesium ions and DNase;
a secondary washing step in which the skin tissue, which has undergone the DNA treatment step, is washed;
a disinfection step in which the skin tissue, which has undergone the secondary washing step, is disinfected using a disinfection solution; and
a tertiary washing step in which the disinfected skin is washed.

2. The bioink of claim 1, wherein the first solution further comprises ethylenediaminetetraacetic acid (EDTA).

3. The bioink of claim 1, wherein, in the first solution, 1 mM or less of the ethylenediaminetetraacetic acid (EDTA) is dissolved and 0.25% or less of the trypsin is dissolved.

4. The bioink of claim 1, wherein the epidermis of the skin tissue is removed in at least one step of the peeling preparation step and the peeling step.

5. The bioink of claim 1, wherein, in the primary washing step, the secondary washing step, and the tertiary washing step, the skin tissues are washed with a buffer solution that comprises phosphate buffered saline (PBS).

6. The bioink of claim 1, wherein the second solution is a buffer solution which comprises 10 mM or less of MgCl₂ and 30 U/mL or less of DNase.

7. The bioink of claim 6, wherein the buffer solution comprises phosphate buffered saline (PBS), and the DNA treatment step is performed at a temperature between 30° C. or higher and 40° C. or lower for at least 20 hours.

8. The bioink of claim 1, wherein the disinfection solution comprises peracetic acid and ethanol.

9. The bioink of claim 1, wherein the buffer solution is phosphate buffered saline (PBS) in which 25 mM or less of the ethylenediaminetetraacetic acid (EDTA) and 1% or less of the non-ionic surfactant are dissolved.

10. The bioink of claim 1, wherein the cell removal step is performed at a temperature between 30° C. or higher and 40° C. or lower for at least 20 hours.

11. The bioink of claim 1, wherein:
the decellularized skin tissue is dissolved in an acetic acid solution along with pepsin to prepare a pre-gel; and
the normal human dermal fibroblasts (NHDF) are mixed with a hydrogel in which the pre-gel is neutralized.

12. A method for preparing artificial skin, which comprises seeding normal human epithelial keratinocytes (NHEK) to the bioink according to claim 1, followed by culturing the same.

13. A method for preparing artificial skin, which comprises:
mixing normal human dermal fibroblasts (NHDF) with a decellularized skin tissue; and
seeding normal human epithelial keratinocytes (NHEK) thereto.

14. The method of claim 13, wherein:
the decellularized skin tissue is in a hydrogel state; and
the normal human epithelial keratinocytes (NHEK) are seeded in a state where the decellularized skin tissue, which is in a hydrogel state where the normal human dermal fibroblasts (NHDF) are mixed, is not shrinked.
